(19) United States
(12) Patent Application Publication
(10) Pub. No.: US 2020/0261668 A1
(43) Pub. Date: Aug. 20, 2020

(54) AN ACTUATOR HOUSING FOR A METERED DOSE INHALER DEVICE

(71) Applicant: KINDEVA DRUG DELIVERY L.P., St. Paul, MN (US)

(72) Inventors: PETER D. HODSON, BREASTON (GB); PHILIP A. JINKS, LOUGHBOROUGH (GB); ADAM J. STUART, LOUGHBOROUGH (GB)

(21) Appl. No.: 15/776,177

(22) PCT Filed: Nov. 15, 2016
(86) PCT No.: PCT/US2016/061973

§ 371 Date: May 15, 2018

(30) Foreign Application Priority Data
Nov. 16, 2015 (GB) 1520174.2

(51) Int. Cl. A61M 15/00

(52) U.S. Cl. CPC A61M 15/0065; A61M 2205/586; A61M 15/0021; A61M 15/009

(57) ABSTRACT

An actuator housing (100) for use with a metered dose inhaler (MDI) device which comprises a housing portion (115) having a nozzle block (130) in which an actuator seat (135) and an actuator nozzle (145) are formed. The housing portion has an axis (120) which is angled with respect to a vertical plane which is perpendicular to a base portion or thumb grip (150) formed at a closed end thereof. A mouthpiece portion (125) is joined to the housing portion which has a roof section (180) and a floor section (125b). The roof section is angled upwards with respect to the floor portion, and provides a cross-piece (185) with which teeth of a patient can engage. Such an actuator housing addresses problems associated with buccal and tongue deposition of medicaments, as a spray of medicament from the actuator nozzle is directed over the tongue and into the airway.

### FIELD OF THE INVENTION

The present invention relates to improvements in or relating to medical actuators and is more particularly concerned with actuators for metered dose inhalers.

### BACKGROUND OF THE INVENTION

All metered dose inhaler (MDI) actuators or devices for pulmonary delivery, whether they be standard “press-and-breathe” designs or designs employing automatic triggering, such as those found in the Autohaler™ or the Easibreathe™ devices, are designed to deliver an aerosol plume to the oral cavity at an angle which is essentially horizontal [Autohaler™ is a trademark of the 3M Corporation and Easibreathe™ is a trademark of Norton Healthcare Limited].

Whereas MDI buccal deposition delivered by an MDI device is widely appreciated, the effect of the horizontal plume delivery characteristic of such devices, in terms of buccal deposition, and in particular, tongue deposition, does not appear to be widely appreciated. In particular, a large proportion of buccal deposition is likely to arise from the tongue being a ballistic target for the plume during delivery to the patient. As a result, the patient may not receive the full dose due to such deposition.

In addition, there is a possibility of the teeth and lips of the patient also providing obstacles for the plume and further locations for deposition if the actuator is not positioned correctly within the mouth of the patient.

EP-A-0475257 discloses an MDI device which dispenses medicaments as aerosols. The device comprises a seat for housing a can containing the constituents of the aerosol, namely, a medicament and a propellant or solvent, and defines an expansion chamber into which the aerosol is discharged through a nozzle, when operated. The dispensed aerosol expands in the expansion chamber circulating with a vortex flow allowing the solvent to evaporate. The flow may continue for a relatively long time, hence enabling only very small particles of the medicament to be drawn into the bronchial tree. There is only minimum aerosol particle deposition on the walls of the oropharyngeal cavity as most of the large aerosol particles are retained within the expansion chamber, and inhalation can continue after the entry of aerosol into the expansion chamber ceases.

Whilst the device is designed to avoid spraying the aerosol directly onto the mucosa of the oropharynx to prevent side effects deriving from direct spray into the mouth, it therefore requires the additional feature of the expansion chamber to allow the propellant or solvent to evaporate leaving only small particles of the medicament to be inhaled with the larger particles being centrifuged onto the walls of the expansion chamber.

WO-A-99/12596 discloses an aerosol nozzle arrangement, for use with the device described in EP-A-0475257, which comprises a T-shaped nozzle which comprises an upper bar and a vertical stem and having a hole through which an aerosol dose is discharged, the aerosol dose being discharged at an angle to a plane normal to an axis of the vertical stem of the nozzle. However, this nozzle arrangement only directs the aerosol dose into the expansion chamber and not into the mouth of a patient.

GB-A-2279879 discloses an MDI device which comprises a housing for a pressurised dispensing container of medicament and a mouthpiece for insertion into the mouth of a patient which is connected by a duct to an outlet of the container. An air inlet is provided for allowing air into the inhaler when the patient applies suction to the mouthpiece. The air inlet is provided at a location axially between the air outlet for the duct and the mouthpiece, a passage connecting the air inlet to a location adjacent the outlet of the duct means. When the patient inhales through the mouthpiece, an airflow is created from the air inlet to the mouthpiece with a component of the airflow being directed away from the mouthpiece towards the outlet of the duct. The mouthpiece is shaped to be inclined at an angle greater than 90 degrees to the axis of the housing so as to present the mouthpiece at a comfortable angle to the patient when the device is held in the hand of the patient.

Such an MDI device, whilst making it more comfortable for the patient, tends to lose a proportion of the medicament in the component of the airflow which is directed away from the mouthpiece to the outlet of the duct.

Pulmonary MDIs are generally operated in a “valve down” orientation. The medicament (formulated as a suspension or solution in a propellant or solvent) is in a canister that has a dispensing valve with a hollow stem that is inserted into an actuator seat. In use, the patient typically squeezes the base of the canister towards the base portion of the actuator to actuate the valve and dispense the medicament as a spray (i.e., pressure actuated dispensing). Most actuators are generally ‘L’-shaped, with a tubular housing for the container forming one branch of the ‘L’ and the mouthpiece forming the other. The base portion is often provided with features to enable it to be gripped by the patient, such as various configurations of thumb grip. The base portion is nevertheless generally planar, defining a base plane, which in use is typically horizontal.

### SUMMARY OF THE INVENTION

In accordance with one aspect of the present invention, there is provided an actuator housing for a metered dose inhaler device, the actuator housing comprising:
a substantially hollow tubular first portion having a first proximal end and a first distal end;
a base portion formed at the first proximal end, the base portion having a base plane;
an actuator seat formed in the base portion;
an actuator nozzle formed in the actuator seat and operable for dispensing a spray of metered fluid; and

a substantially hollow second portion having a second proximal end and a second distal end and defining a mouthpiece, the second proximal end being located adjacent the first proximal end and the second distal end defining an end of the mouthpiece, the substantially hollow second portion defining a roof section and a floor section which extend from the second proximal end to the second distal end of the mouthpiece, the roof section having an outer profile angled upwardly relative to the base plane and the floor section being in juxtaposition with the base portion and having an outer profile substantially parallel with the base plane.

The use of the term “angled” herein relates to a deviation in direction from a specified reference direction, such deviation being an acute angle.

By having an upwardly angled roof portion of the mouthpiece, a patient is provided with a location over which at least the lips and preferably the teeth need to be placed thereby opening the mouth wider; additionally, in some embodiments the actuator housing design serves to generate an upwardly directed plume of medicament when dispensed from a canister of medicament inserted into the actuator housing.

In one embodiment, the outer profile of the roof section defines an upward angle between 10 degrees and 40 degrees relative to the base plane. Preferably, the roof section defines an upward angle which is substantially 30 degrees relative to the base plane.

Ideally, only the roof section of the mouthpiece is modified. This modification opens the airway as the lower teeth and lips tend not to interfere with the projection of the spray.

In a modified embodiment, the actuator nozzle is upwardly angled with respect to the base plane to produce an upwardly angled spray with respect to the base plane. In an embodiment, the upward angle of the actuator nozzle with respect to the base plane is in the range of between 10 degrees and 50 degrees, preferably between 20 degrees and 40 degrees, more preferably between 25 degrees and 35 degrees, and most preferably substantially 30 degrees.

By having both the angled roof portion and the actuator nozzle angled upwardly, it is possible to substantially reduce buccal and tongue deposition of the medicament being dispensed.

In addition, the actuator seat may also be angled with respect to the base plane to produce the upwardly angled actuator nozzle. This simplifies the manufacturing process for the actuator housing whilst providing the benefits of being able to generate an upwardly directed plume of medicament.

The substantially hollow second portion may be substantially conical in shape, with the second distal end having an end face which has a greater diameter than the second proximal end thereof.

The actuator housing may further comprise a cross-piece formed adjacent a first end of the outer profile of the roof section which is adjacent the second proximal end of the mouthpiece.

Advantageously, the provision of the cross-piece provides a locator for the teeth of a patient when using an MDI device including such an actuator housing.

In accordance with another aspect of the present invention, there is provided an actuator housing for a metered dose inhaler device, the actuator housing comprising:
a substantially hollow tubular first portion having a first proximal end and a first distal end and a first portion axis extending from the first distal end to the first proximal end;
a base portion formed at the first proximal end;
an actuator seat formed in the base portion;
an actuator nozzle formed in the actuator seat and operable for dispensing a spray of metered fluid; and

a substantially hollow tubular second portion having a second proximal end and a second distal end defining a mouthpiece, the second proximal end being located adjacent the first proximal end and the second distal end defining an end face of the mouthpiece, the substantially hollow tubular second portion having a second portion axis extending from a centre of the actuator nozzle to a centre of the end face of the mouthpiece;

wherein the actuator nozzle defines a spray axis which is angled upwardly in the distal direction relative to a plane perpendicular to the first portion axis.

By having a spray direction which is angled upwardly due to the orientation of the spray axis, the spray is directed over the tongue of a patient thereby reducing tongue deposition.

In one embodiment, the second portion axis is angled upwardly relative to a plane perpendicular to the first portion axis. The spray direction may be aligned with the second portion axis. Alternatively, the spray axis may be upwardly angled in the distal direction relative to the second portion axis.

The spray axis may be angled upwardly in the distal direction between 10 degrees and 50 degrees relative to the plane perpendicular to the first portion axis. In one embodiment, the spray axis is angled upwardly substantially 30 degrees relative to the plane perpendicular to the first portion axis.

In accordance with a further aspect of the present invention, there is provided an actuator housing for a metered dose inhaler device, the actuator housing comprising:
a generally tubular substantially hollow first portion having a first distal end and a first proximal end and a first portion axis extending from the first distal end to the first proximal end;
a base portion formed at the first proximal end;
an actuator seat formed in the base portion;
an actuator nozzle formed in the actuator seat and operable for dispensing a spray of metered fluid; and

a generally tubular substantially hollow second portion having a second proximal end and a second distal end defining a mouthpiece, the second proximal end being located adjacent the first proximal end and the second distal end defining an end of the mouthpiece, the generally tubular substantially hollow second portion having a second portion axis extending from a centre of the actuator nozzle to a centre of the end of the mouthpiece, the end of the mouthpiece having an open face defining a mouthpiece face plane which is angled relative to a plane perpendicular to the second portion axis.

Preferably the mouthpiece face plane is angled such that the mouthpiece roof portion extends distally, in the second portion axial direction, further than its floor portion.

By having an angled mouthpiece face plane orientated thus, a patient is encouraged to angle a MDI device of which the actuator housing forms a part into a position where both the upper and lower lips can be sealed against the mouthpiece during inhalation of a dispensed medicament and at an angle at which the emerging spray tends to be directed more upwardly over the tongue into the oral cavity.

The mouthpiece face plane may be at an angle between 10 degrees and 50 degrees relative to the plane perpendicular to the second portion axis, preferably 30 degrees. In a preferred embodiment, the spray direction is angled upwardly substantially 30 degrees in the distal direction relative to the plane perpendicular to the first portion axis.

Alternatively, the actuator nozzle defines a spray axis which is angled upwardly in the distal direction relative to the second portion axis.

With the combination of the angled mouthpiece face plane and the angled spray direction, the delivery of a medicament from an MDI device incorporating such an actuator housing is more likely to be directed above and over the tongue of the patient.

In addition to having an angled end face at the second distal end of the mouthpiece, the generally tubular substantially hollow second portion of the actuator housing may further comprise a first region and a second region formed on the external surface, the first region being closer to the distal end than the second region and being configured to define a guidance region, the second region being located adjacent the proximal end and being configured to define a sealing region.

By providing a guidance region for a patient over which the teeth are to be located and a sealing region against which the lips can be sealed, a patient is intuitively encouraged to put the mouthpiece far enough into the mouth so that the lips make a comfortable seal against the sealing region whilst ensuring that a medicament spray is not obstructed by the teeth, that the mouth is properly open and that the airway is clear.

In one manifestation, the generally tubular substantially hollow second portion of the actuator may comprise a plurality of flutes formed on an external surface of the mouthpiece and located adjacent to the mouthpiece face plane.

The provision of the flutes ensures that a patient has to align the lips in a position to form a seal when inhaling the dispensed medicament.

Preferably, each flute extends from the mouthpiece face plane in a direction substantially parallel to the base portion.

Sealing of the lips of a patient over the flutes to a position on the mouthpiece beyond them ensures that the mouthpiece has correctly been introduced into the mouth. Because the lips are only a certain distance forward of the teeth, then having extended flutes ensures that the teeth must be around the mouthpiece, rather than be obstructively positioned in front of its face plane, when the lips are sealed on the mouthpiece beyond the flutes.

An annular projection may be provided and located on an external surface of the mouthpiece, the plurality of flutes being located between the mouthpiece face plane and the annular projection.

The addition of such an annular projection provides a stop for the lips of the patient ensuring correct positioning of the MDI device of which the actuator housing forms a part.

As an alternative to the flutes, a plurality of apertures may be formed in the mouthpiece adjacent the mouthpiece face plane.

These apertures have the same function as the flutes, namely to ensure that a seal is only created between the lips of the patient and the mouthpiece when the mouthpiece is in a suitable position to ensure that the MDI device of which the actuator housing forms a part is correctly located and aligned to at least reduce the buccal and tongue deposition.

The plurality of apertures may define at least one ring around the mouthpiece.

Sealing of the lips of a patient over the apertures to a position on the mouthpiece beyond them ensures that the mouthpiece has correctly been introduced into the mouth and that the teeth have been forced around the mouthpiece, thereby reducing the risk of unwanted premature deposition of the medicament on the teeth.

An annular projection may be provided and located on an external surface of the mouthpiece, the plurality of apertures being located between the mouthpiece face plane and the annular projection.

The addition of such an annular projection provides a stop for the lips of the patient ensuring correct positioning of the MDI device of which the actuator housing forms a part.

As an alternative to the plurality of apertures, a plurality of depressions may be provided on the mouthpiece adjacent the mouthpiece face plane. The plurality of depressions may define at least one ring around the mouthpiece.

For the same reason as with the provision of holes and flutes, an annular projection may be provided which is located on an external surface of the mouthpiece, the plurality of depressions being located between the mouthpiece face plane and the annular projection.

In another alternative, a plurality of rib elements may be formed on an external surface of the mouthpiece adjacent the mouthpiece face plane. Each rib element may be spaced from the mouthpiece face plane and extends in a direction which is substantially parallel with the base portion. The plurality of rib elements may define at least one ring around the mouthpiece.

Sealing of the lips of a patient over and beyond the rib elements ensures that the mouthpiece has correctly been introduced into the mouth.

As before, an annular projection may be located on the external surface of the mouthpiece, the plurality of rib elements being located between the mouthpiece face plane and the annular projection.

In a further alternative, the mouthpiece may include at least one annular projection formed on an external surface thereof, each annular projection being spaced from the mouthpiece face plane. Each annular projection may be aligned to be substantially parallel with the mouthpiece face plane. In one embodiment, there is a single annular projection which extends around an external surface of the mouthpiece. If a single annular projection is provided, it may be shaped to match the shape of the lips of a user.

This shaping of the single annular projection provides an abutment or stop against which the patient can place the lips to ensure correct positioning of the MDI device in the mouth.

In another embodiment, said at least one annular projection comprises three annular projections each of which extends around the external surface of the mouthpiece. Each of the three annular projections may be divided into a plurality of portions.

This has the advantage of encouraging the patient to place their lips beyond the annular projections in order to achieve a good seal with the region of the mouthpiece beyond the annular projections. By so doing, the patient also tends to have to place their teeth around the mouthpiece rather than in a position which obstructs the open end thereof.

In addition to the embodiments in which the mouthpiece face plane is angled with respect to the first portion axis, the actuator nozzle may be upwardly angled with respect to the base portion to produce an upwardly angled spray with respect thereto.

In accordance with yet another aspect of the present invention, there is provided a metered dose inhaler device comprising a canister having a metering valve, and an actuator housing as described above, the metering valve being operable for engaging with the actuator seat formed in the base portion of the actuator housing.

Typically, such dispensing devices are used valve down and the valve is gravity-fed, so the valve does not have a dip tube. In dispensing devices that are used valve up, a dip tube is required. A dip tube can present a problem when dispensing formulations because of loss of prime in propellant-based formulations and/or due to inhomogeneity of the drug suspension which could take several actuations to advance up the tube.

Naturally, such a metered dose inhaler device has the advantages conferred by the actuator in that the plume of medicament dispensed from the canister via the metering valve is more likely to be effectively angled upwards with respect to the horizontal, and/or is less likely to be obstructed by the upper front teeth, and therefore substantially reduces the loss of medicament received by the patient due to buccal and tongue deposition as well as deposition on the teeth and lips.

In accordance with a further aspect of the present invention, there is provided a method of manufacturing a metered dose inhaler device comprising an actuator configured to deliver an aerosol spray from a nozzle orifice having an axis and to predispose a user of the metered dose inhaler device to direct the nozzle orifice axis towards the roof of the user's mouth. In each of the foregoing embodiments users have a predisposition to direct the nozzle thus.

In accordance with an additional further aspect of the present invention, there is provided a method of treatment of a pulmonary condition in a human patient, the method comprising providing a metered dose inhaler device comprising an actuator with a mouthpiece, the actuator configured to deliver an aerosol spray from a nozzle orifice having an axis and to predispose a patient to direct the nozzle orifice axis towards the roof of the patient's mouth, the patient inserting the mouthpiece into their mouth and actuating the metered dose inhaler device while inhaling.

In accordance with yet an additional further aspect of the present invention, there is provided a method of treatment of a pulmonary condition in a human patient, the method comprising providing a metered dose inhaler device comprising an actuator with a mouthpiece, the actuator configured to deliver an aerosol spray from a nozzle orifice having an axis and to predispose a patient to direct the nozzle orifice axis towards the roof of the patient's mouth, the patient inserting the mouthpiece into their mouth, aligning the nozzle orifice towards the roof of their mouth, and actuating the metered dose inhaler device while inhaling.

In accordance with a still further aspect of the present invention, there is provided a method of treatment of a pulmonary condition in a human patient, the method comprising providing a metered dose inhaler device comprising an actuator with a mouthpiece, the actuator configured to deliver an aerosol spray from a nozzle orifice, placing at least a portion of the actuator mouthpiece in the mouth of the patient, directing the nozzle orifice axis towards the roof of the mouth, actuating the metered dose inhaler device while inhaling, so as to reduce ballistic tongue deposition of the resulting aerosol plume as compared to the amount of ballistic tongue deposition that would result if the nozzle orifice axis was directed into the oral cavity at an essentially horizontal angle. In a preferred aspect of this method, the patient places their teeth around the outside of the mouthpiece prior to actuating and inhaling.

The present invention also provides for a kit comprising a metered dose inhaler device comprising an actuator with a mouthpiece and a set of instructions to a patient to use the device according to any of the methods described above.

### BRIEF DESCRIPTION OF THE DRAWINGS

For a better understanding of the present invention, reference will now be made, by way of example, to the accompanying drawings in which:

FIG. 1a illustrates a sectioned side view of a first type of conventional actuator;

FIG. 1b illustrates a front view of the actuator of FIG. 1a;

FIG. 2a illustrates a sectioned side view of a second type of conventional actuator;

FIG. 2b illustrates a front view of the actuator of FIG. 2a;

FIG. 3a illustrates a sectioned side view of a first embodiment of an actuator in accordance with the present invention;

FIG. 3b illustrates a front view of the actuator of FIG. 3a;

FIG. 4 illustrates a sectioned side view of the actuator of FIGS. 3a and 3b, including a canister;

FIG. 5a illustrates a sectioned view of a second embodiment of an actuator in accordance with the present invention;

FIG. 5b illustrates a front view of the actuator of FIG. 5a;

FIG. 6 illustrates a sectioned side view of a third embodiment of an actuator in accordance with the present invention;

FIG. 7a is similar to FIG. 6 but illustrates a sectioned side view of another embodiment of an actuator in accordance with the present invention;

FIG. 7b illustrates a side view of the actuator of FIG. 7a;

FIG. 8 is similar to FIG. 7b but illustrates a side view of another embodiment of an actuator in accordance with the present invention;

FIG. 9 is similar to FIG. 7b but illustrates a side view of another embodiment of an actuator in accordance with the present invention;

FIG. 10 is similar to FIG. 7b but illustrates a side view of another embodiment of an actuator in accordance with the present invention;

FIG. 11 is similar to FIG. 7b but illustrates a side view of another embodiment of an actuator in accordance with the present invention;

FIG. 12 is similar to FIG. 7b but illustrates a side view of another embodiment of an actuator in accordance with the present invention;

FIG. 13a is similar to FIG. 7a but illustrates a sectioned side view of another embodiment of an actuator in accordance with the present invention; and

FIG. 13b illustrates a front view of the actuator of FIG. 13a.

### DESCRIPTION OF THE INVENTION

The present invention will be described with respect to particular embodiments and with reference to certain drawings but the invention is not limited thereto. The drawings described are only schematic and are non-limiting. In the drawings, the size of some of the elements may for illustrative purposes be exaggerated and not drawn to scale.

It will be understood that the terms “vertical”, “horizontal”, “top”, “bottom”, “above”, “below”, “left”, “right” etc. as used herein refer to particular orientations of the Figures and these terms are not limitations to the specific embodiments described herein.

MDI actuators generally comprise a canister-retaining or tubular housing portion and a tubular mouthpiece portion, the tubular mouthpiece portion being angled with respect to an axis extending through the tubular housing portion. At a closed bottom end of the tubular housing portion sits a nozzle block that comprises a stem socket and an exit orifice or actuator nozzle. At the bottom of the actuator, a thumb grip is provided. The tubular mouthpiece portion may have a circular, elliptical or oblong cross-section. It has an opening that is generally greater than 13 mm in its shortest internal and external dimensions, or generally greater than 20 mm in its longest internal and external dimensions, or generally greater than 300 mm² in internal and external areas.

The term “oblong” as used herein refers to a shape which deviates from a square or circular form principally by elongation in one dimension.

In normal operation of an MDI device (an MDI actuator housing and a canister of medicament), a plume of medicament is produced from the exit orifice or actuator nozzle into the tubular mouthpiece portion and is inhaled by a patient through the tubular mouthpiece portion. However, as described above, there tends to be substantial and undesirable deposition of the medicament in the buccal cavity when the medicament is dispensed from a conventional device.

The term “spray axis” as used herein refers to an imaginary line or ray that originates from a centre of the exit orifice or actuator nozzle and extends to or through the end face of the mouthpiece. In some embodiments described herein, the spray axis extends to or through a centre of the end face of the mouthpiece. In the figures, a spray axis is depicted by the arrows 55, 155, 955, and 1155.

A spray can be directed as a plume aligned along the spray axis.

Experiments were conducted to show that a large proportion of buccal deposition is likely to arise from the tongue being a ballistic target for a plume emitted from an MDI device. Using public domain magnetic resonance imaging (MRI) scans of the head of an individual to identify the position of the tongue during delivery of a medicament using MDI devices and in addition performing measurements using a cut-away actuator, it has become apparent that most of the deposition found in the mouth is likely to be on the tongue rather than on the back of the throat. This is in contrast to a common misconception within both the clinical and the technical MDI fields. There is therefore potential for a large improvement in MDI drug therapy efficiency, simply by the avoidance of tongue deposition.

The present invention provides for a number of actuator embodiments in which the MDI spray is effectively emitted upwards at an angle into the oral cavity to reduce substantially or eliminate ballistic tongue deposition. There are two main ways to angle the spray upwards into the mouth, the first being to encourage the patient (or user of the MDI device) to tilt the top of the canister away from the face, and the second being to angle the exit orifice or actuator nozzle upwards relative to the canister and/or the mouthpiece. In addition, a combination of upwardly angled exit orifice or actuator nozzle (and/or mouthpiece) and encouraging the patient to tilt the top of the canister away from the face is also beneficial for the reduction of ballistic tongue deposition.

In the embodiments described below, the actuators encourage the patient to tilt the actuator away from the face by making the mouthpiece more comfortable or convenient to use at an angled orientation. In one embodiment, the mouthpiece of the actuator is angled upwards to provide an upwardly directed spray. In other embodiments, the end of the mouthpiece of the actuator is angled to encourage the patient to aim the mouthpiece upwards to provide an upwardly directed spray in use. In a further embodiment, the nozzle block is angled so that the exit orifice or actuator nozzle produces an upwardly directed spray. In addition, combinations of these embodiments are also possible as will be described in more detail below.

Ideally, in some embodiments, the actuator may also provide for a location position for at least the teeth on an external surface thereof which is both comfortable and intuitive for a patient using the MDI device of which the actuator forms a part.

Furthermore, in some embodiments, the actuator housing may provide features on or in the external surface of the mouthpiece which provide a topography or texture on which the patient's lips can only uncomfortably rest and/or which make it difficult to form a seal between the lips and that region of the mouthpiece. By providing a smooth region of external surface further along the mouthpiece, away from its open end, onto which the lips can intuitively and obviously seal, the patient may be encouraged to push the mouthpiece further into their oral cavity. Reaching the smooth sealing region with their lips in this way tends to force them to place their teeth around the outside of the mouthpiece instead of leaving them in the path of the medicament spray which will emerge from its open end.

As mentioned above, experiments were conducted using a conventional MDI actuator housing with a cut-away back section to demonstrate that ballistic tongue deposition can be avoided by designing an actuator which fires upwards into the oral cavity at an angle from horizontal of approximately 30 degrees, so that the spray passes over and above the tongue and has the opportunity of evaporating fully within the full volume of the oral or buccal cavity. In vivo measurements were performed using a cut away actuator showing the proximity of the tongue to the outlet of the actuator. Microscopy samples of the emitted dose of MDI devices were also collected by placing a slide at a distance of 5 cm away from the actuator mouthpiece, and it became apparent that the tongue, which is directly in line with the emitted spray and far closer to the mouthpiece (typically between 1.5 cm and 2.5 cm) in the real life situation than the slide at 5 cm, presents a considerable target for unwanted spray impact and deposition.

In one embodiment, the spray emitted from the actuator is at an angle upwards from horizontal of between 20 and 50 degrees and preferably of around 30 degrees.

Another major factor affecting MDI delivery efficiency and buccal deposition is the proximity of the teeth, in particular the upper front teeth, to the actuator outlet, during inhaler use. There appears to be a large degree of variation as to the way that the manufacturers of MDI devices recommend that they be used. For example, some manufacturers instruct the patient to place the teeth around the mouthpiece, with others instructing the patient to place the lips around the mouthpiece but giving no instruction as to the position of the teeth. Unfortunately, manufacturers' instructions are renowned for not always being carefully studied or followed, and thus devices should preferably always be designed to intuitively encourage correct use.

By employing the cut-away actuator, it has been confirmed that if only the lips are placed around the mouthpiece and not also the teeth, then the teeth themselves can create a large obstacle to MDI delivery with high levels of ballistic impact of the emitted spray on the teeth as a consequence.

In addition to the need for an MDI actuator housing that overcomes the issue of tongue drug impaction arising from the horizontal spray delivery of current actuators, by having an upward angle of spray delivery, there is also a need for an actuator to be designed in such a way that the patient will intuitively place the teeth in the correct position, out of the line of fire of the emitted spray and on the outside of the actuator.

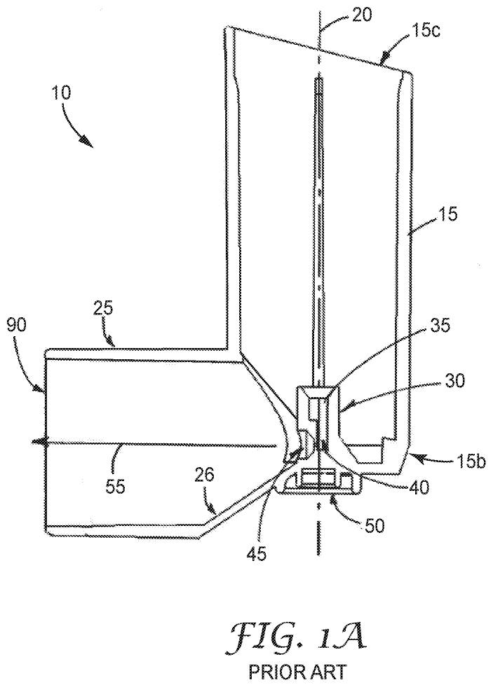

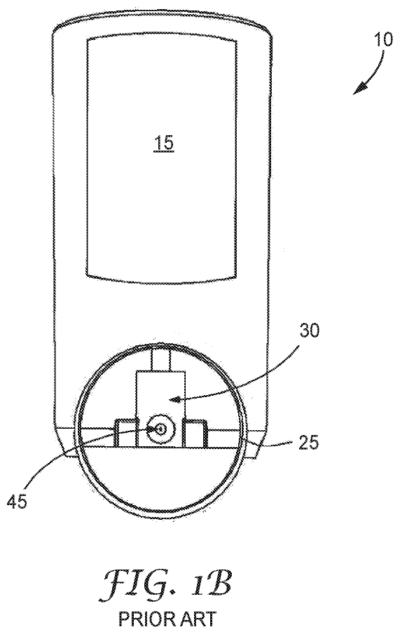

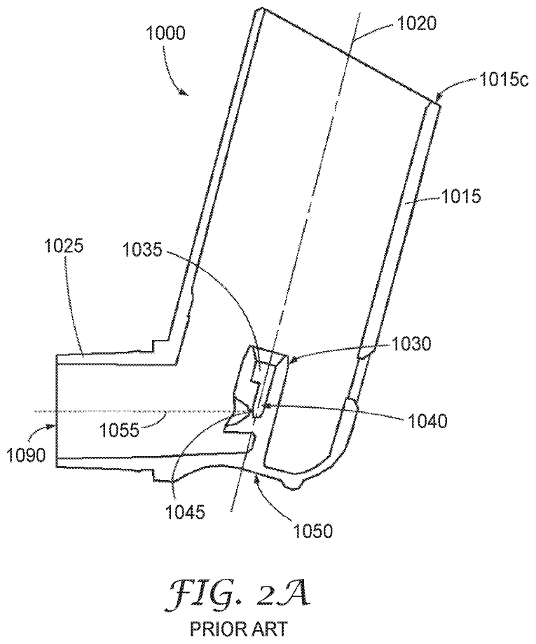

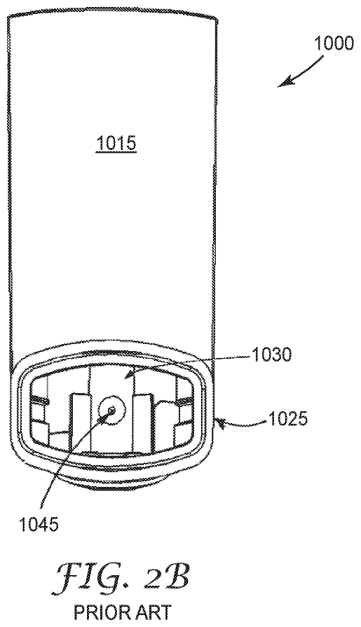

Before describing the embodiments in accordance with the present invention, one embodiment of a conventional MDI actuator housing will be described with reference to FIGS. 1 and 2.

FIG. 1a illustrates a side sectioned view through a conventional MDI actuator housing 10 which comprises a substantially hollow tubular housing portion 15 having an axis 20 which extends therethrough and a substantially hollow tubular mouthpiece portion 25. The tubular housing portion 15 has an open or distal end 15c and a closed or proximal end 15b, the closed or proximal end being located at a bottom or lower end of the tubular housing portion 15 and forming a base portion as shown in the Figure. Within the closed or proximal end 15b sits a nozzle block 30 which comprises an actuator seat or stem socket 35 in fluid communication with a sump region 40 and an exit orifice or actuator nozzle 45. On an external surface of the closed or proximal end 15b of the tubular housing portion 15, a thumb grip 50 is provided in the base portion which is substantially perpendicular to the axis 20.

The tubular mouthpiece portion 25 comprises a proximal end which adjoins the closed or proximal end 15b of the tubular housing portion 15, and is connected via a partially conical transition 26 to the base portion. The tubular mouthpiece portion 25 also has an open or distal end which comprises a mouthpiece end face 90, and the tubular mouthpiece portion extends from its proximal end in a direction towards its distal end and is substantially aligned with an axis 55 which extends from a centre of the exit orifice or actuator nozzle 45 to a centre of the mouthpiece end face 90. In this embodiment, the spray is directed from the exit orifice or actuator nozzle 45 in the direction of the axis 55 as indicated by the arrow. The mouthpiece end face 90 defines a mouthpiece face plane, which, in FIG. 1a, is substantially parallel with the axis of the housing portion 15 and perpendicular to a plane in which the axis of the mouthpiece portion 25 lies. In this embodiment, the mouthpiece face plane 90 is also perpendicular to a plane in which the base portion lies.

FIG. 1b shows a front view of the actuator housing of FIG. 1a, and, features described above with reference to FIG. 1 a, that are visible in this Figure, are indicated using the same reference numerals.

It will readily be understood, from the description of FIGS. 2a and 2b below, that a canister containing a medicament can be inserted into the actuator housing in a similar way with the spray being directed substantially along the axis 55.

FIG. 2a illustrates a side sectioned view through a conventional MDI actuator housing 1000 which comprises a substantially hollow tubular housing portion 1015 having an axis 1020 which extends therethrough and a substantially hollow tubular mouthpiece portion 1025. The tubular housing portion 1015 has a nozzle block 1030 which comprises an actuator seat or stem socket 1035 in fluid communication with a sump region 1040 and an exit orifice or actuator nozzle 1045. The tubular mouthpiece portion 1025 also has an open or distal end which comprises a mouthpiece end face 1090, and the tubular mouthpiece portion extends from its proximal end in a direction towards its distal end and is substantially aligned with an axis 1055 which extends from a centre of the exit orifice or actuator nozzle 1045 to a centre of the mouthpiece end face 1090. In this embodiment, although not shown, the spray is directed from the exit orifice or actuator nozzle 1045 in the direction of the axis 1055.

FIG. 2b shows a front view of the actuator shown in FIG. 2a, and, features described above with reference to FIG. 2a, that are visible in this Figure, are indicated using the same reference numerals.

A canister containing a medicament (not shown) is mounted within the tubular housing portion 1015 so that a valve stem thereof is located within the nozzle block 1030, and in particular, within the actuator seat or stem socket 1035. An upper end of the canister extends beyond the open or distal end 1015c of the tubular housing portion 1015. Downward pressure on the upper end of the canister activates a valve associated with the valve stem to release a predetermined amount of medicament into the sump region 1040 and through to the exit orifice or actuator nozzle 1045 to generate a spray (not shown) which is directed through the tubular mouthpiece portion 1025 in the direction of the axis 1055. As described above, the base portion and the thumb grip 1050 lie in a plane which provides a horizontal reference for the angle of the generated spray which is, for example, substantially 0 degrees as indicated by dotted line 1055.

In accordance with one embodiment of the present invention, an MDI actuator housing has been designed such that, when inserted into the mouth, the upper teeth rest comfortably and intuitively in a recess formed between an upwardly angled top section of the mouthpiece portion and the tubular housing portion of the actuator.

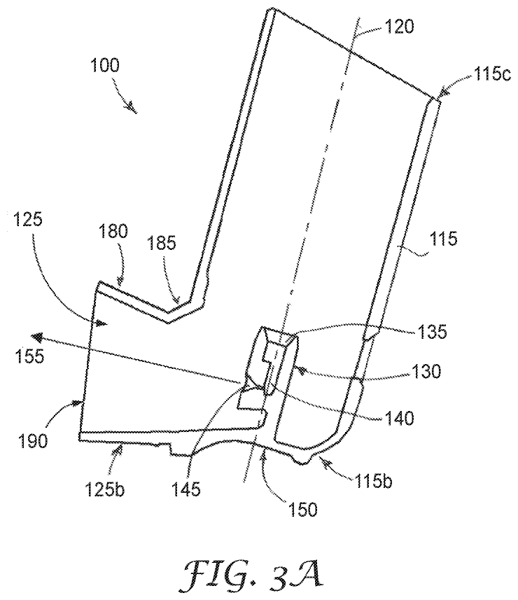

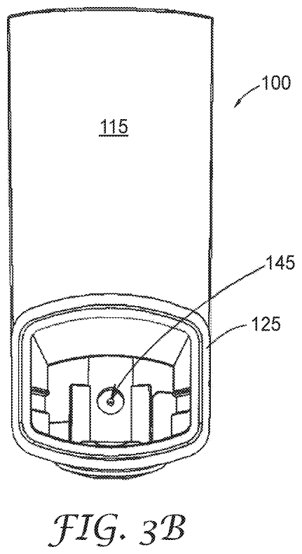

In FIG. 3a, a side sectioned view through an MDI actuator housing 100 is shown which comprises a substantially hollow tubular housing portion 115 having an axis 120 extending therethrough and a substantially hollow mouthpiece portion 125. The tubular housing portion 115 has an open or distal end 115c and a closed or proximal end 115b, the closed or proximal end being located at a bottom or lower end of the tubular housing portion 115 and forming a base portion. Within the closed or proximal end 115b sits a nozzle block 130 which comprises an actuator seat or stem socket 135 in fluid communication with a sump region 140 and an exit orifice or actuator nozzle 145. On an external surface of the closed or proximal end 115b forming the base portion, a thumb grip 150 is provided.

The mouthpiece portion 125 comprises a proximal end which adjoins the closed or proximal end 115b of the tubular housing portion 115. The tubular mouthpiece portion 125 also has an open or distal end which comprises a mouthpiece end face 190, and the mouthpiece portion extends from its proximal end in a direction towards its distal end. The mouthpiece end face 190 defines a mouthpiece face plane.

In this embodiment, the mouthpiece portion 125 has a roof section 180 and a floor section 125b, the roof section 180 being angled upwards with respect to the floor section 125b. The exit orifice or actuator nozzle 145 is angled upwards relative to a line (not shown) between a centre of the exit orifice or actuator nozzle and a centre of the mouthpiece end face plane 190.

In addition, the roof section 180 of the mouthpiece portion 125 is shaped to provide a cross-piece 185 on which a patient can position the upper teeth during use of an MDI device including the actuator housing 100. This cross-piece 185 has the effect that the teeth are clear of the spray of medicament being dispensed.

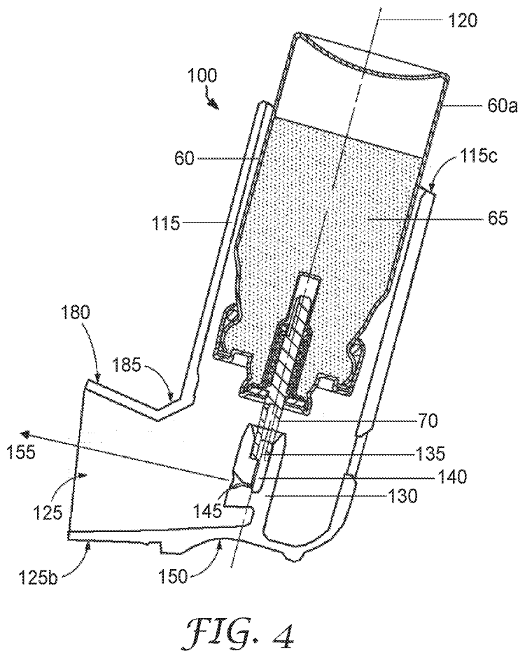

In this Figure, the tubular housing portion 115 is shown angled with respect to a vertical plane which is perpendicular to a plane in which the base portion and associated thumb grip 150 lie. The exit orifice or actuator nozzle 145 is angled upwards with respect to the thumb grip 150 and base portion to provide a spray axis (155) having an upward angle in the distal direction of between 10 degrees and 50 degrees, more preferably 30 degrees, with respect to the plane in which the base portion and associated thumb grip 150 lie. This upward angling of the spray has the effect of projecting the spray away from and over and above the tongue and high into the oral or buccal cavity so that ballistic impaction on the tongue and teeth is substantially reduced or even eliminated. It will be appreciated that, for the actuator used in the inhaler of FIG. 4, the spray will be in the form of a plume. Consequently, the upward angle of the actuator nozzle 145 is selected to project well clear of the roof section 180. The upward angle of the roof section is suitably equal to or greater than the upward angle of the nozzle section.

FIG. 3b shows a front view of the actuator of FIG. 3a, and, features described above with reference to FIG. 3a, that are visible in this Figure, are indicated using the same reference numerals.

FIG. 4 shows a canister 60 containing a medicament 65 mounted within the tubular housing portion 115 of the actuator housing 100 of FIGS. 3a and 3b so that a valve stem 70 thereof is located within the nozzle block 130, and in particular within the actuator seat or stem socket 135. An upper end 60a of the canister extends beyond the open or distal end 115c of the tubular housing portion 115. Downward pressure on the upper end 60a of the canister activates a valve associated with the valve stem 70 to release a predetermined amount of medicament into the sump region 140 and through to the exit orifice or actuator nozzle 145 to generate a spray (not shown) which is directed through the tubular mouthpiece portion 125 in a plume centred around spray axis 155.

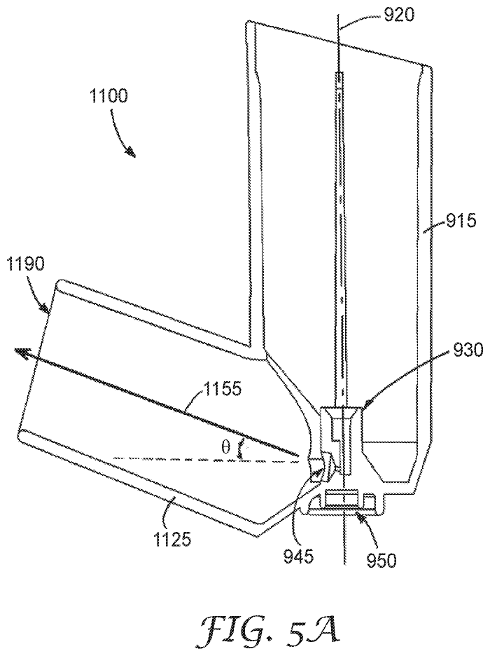

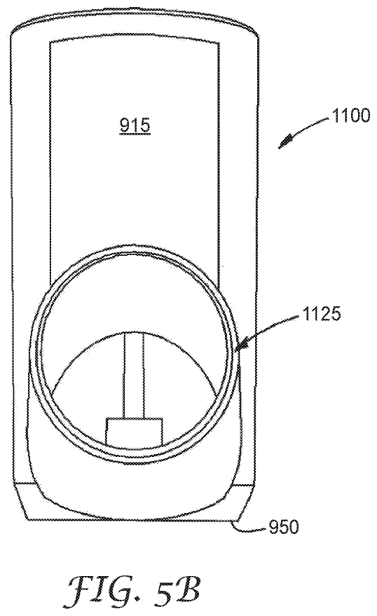

FIG. 5a illustrates a sectioned side view of a second embodiment of an MDI actuator housing 1100 in accordance with the present invention, and FIG. 5b illustrates a front view thereof. The actuator housing 1100 has a combination of a tubular mouthpiece portion 1125 which is angled upwards with respect to a horizontal plane, indicated by the dotted line, which is perpendicular to axis 920 of the housing portion 915 and which is substantially parallel to a horizontal, or base, plane in which thumb grip 950, and hence a base portion of the tubular housing portion 915, lie. Within the closed or proximal end of the tubular housing portion 915 sits a nozzle block 930 having an exit orifice or actuator nozzle 945, the nozzle block 930 being aligned with the axis 920. In this case, the actuator nozzle 945 is located nearer to the bottom or base portion of the tubular housing portion 915. The tubular mouthpiece portion 1125 extends from the closed or proximal end of the tubular housing portion 915 in a direction which is upwardly angled with respect to the base plane.

The exit orifice or actuator nozzle 945 is also angled upwardly with respect to the horizontal plane in which the base portion and thumb grip 950 lie so that a spray can be dispensed therefrom along a spray axis (indicated by arrow 1155) forming an angle θ with respect to the horizontal plane (shown as a dashed line) which is substantially parallel to the horizontal plane in which the thumb grip lies. Angle θ may be in the range of between 20 degrees and 50 degrees, more preferably between 20 degrees and 40 degrees, and most preferably between 25 degrees and 35 degrees. In particular, an upward angle of 30 degrees has been found to be particularly effective in reducing buccal deposition, and in particular, tongue deposition. It is to be understood that the term “horizontal plane” as used herein relates to the particular orientation as shown in FIG. 5a.

This embodiment not only causes the patient to angle the tubular mouthpiece 1125 in the mouth with the housing portion angled away from the face when using an MDI device including such an actuator housing, but the upward angling of the spray ensures that the spray passes over the tongue of the patient and into the airway.

The lowering of the exit orifice or actuator nozzle 945 reduces deposition of the medicament on the uppermost inner surface of the tubular mouthpiece portion 1125. Ideally, the spray is a plume aligned and centred along a spray axis 1155 which is formed as a line (or ray) originating from the centre of the exit orifice or actuator nozzle 945 and passing through a centre of the end face of the tubular mouthpiece portion 1125.

As shown, the exit orifice or actuator nozzle 945 produces an upwardly directed spray, as indicated by arrow 1155, which is at an angle θ to the horizontal plane, and which exits the tubular mouthpiece portion 1125 substantially at a centre of end face or mouthpiece face plane 1190.

The combination of an upwardly angled tubular mouthpiece portion and an upwardly angled exit orifice or actuator nozzle helps to ensure that the spray from the exit orifice or actuator nozzle passes over the tongue with minimum deposition thereon.

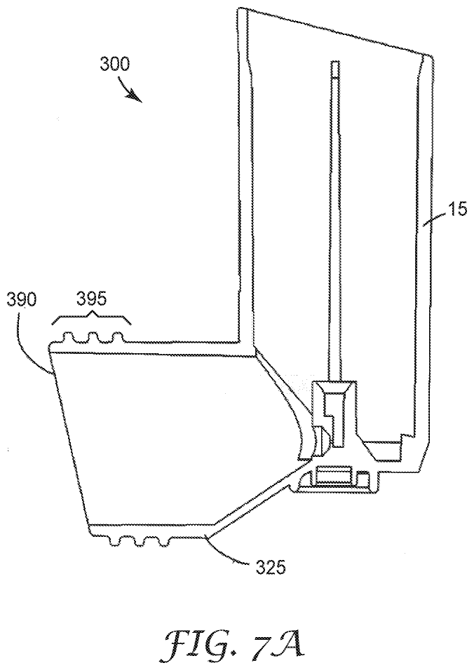

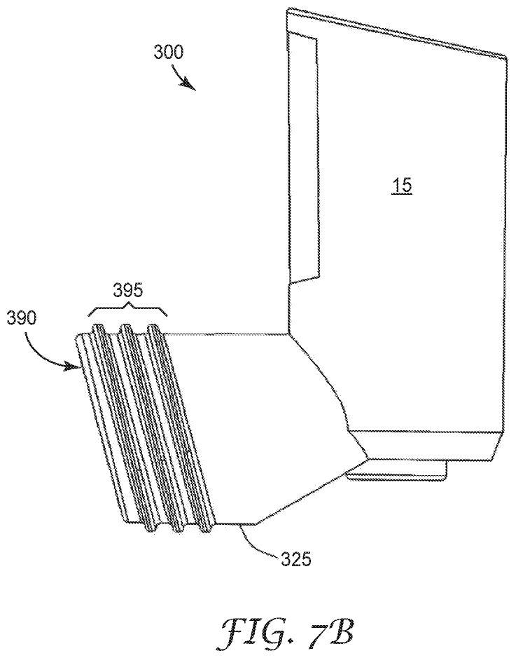

In modifications of the embodiment described with reference to FIG. 5a, the tubular mouthpiece portion may be lengthened and at least one annular ring may be added as described below with reference to FIGS. 7a, 7b and 10 to 12. The addition of at least one annular ring provides an indication that the device needs to be inserted deep into the mouth and that the teeth need to rest on the outside of the tubular mouthpiece portion.

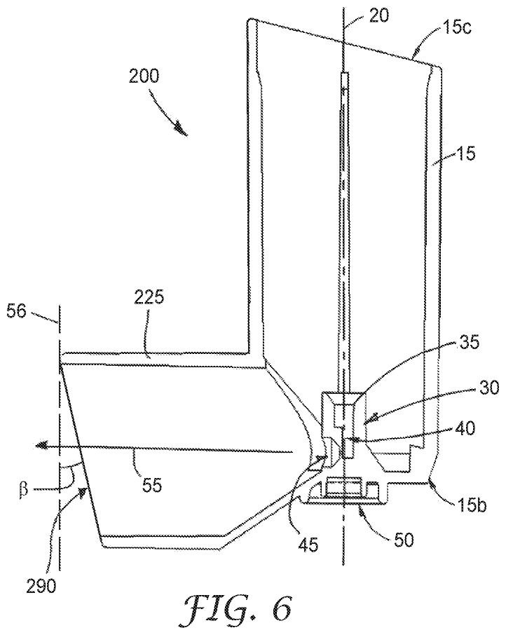

FIG. 6 illustrates a third embodiment of an MDI actuator housing in accordance with the present invention. The actuator housing 200 is similar to the conventional actuator housing 10 shown in FIGS. 1a and 1b, but the tubular mouthpiece portion 225 has an end face 290 defining a mouthpiece face plane which is not perpendicular to the axis of the tubular mouthpiece portion 225. Elements that have previously been described with reference to FIGS. 1a and 1b have the same reference numerals and will not be described again here.

The tubular mouthpiece portion 225 has a spray axis 55 which extends from a centre of the exit orifice or actuator nozzle 45 to a centre of the end face 290. The end face or mouthpiece face plane 290 is angled with respect to the axis 20 of the tubular housing portion 15 and to a plane 56 perpendicular to the axis 55 of the tubular mouthpiece portion 225. In FIG. 6 the angle between the plane 56 perpendicular to the axis 55 and the mouthpiece face plane 290 is shown as 13. Typically, the angle β is between 10 and 50 degrees, preferably substantially 30 degrees. The angle of the mouthpiece face plane with respect to the axis 20 of the tubular housing portion 15 is typically between 10 and 50 degrees, preferably substantially 30 degrees. As shown, in this embodiment where the mouthpiece portion axis 55 is perpendicular to the axis 20 of the tubular housing portion 15, the angles of the end face or mouthpiece face plane 290 with respect to the axis of the tubular housing portion and with respect to a plane perpendicular to the axis of the tubular mouthpiece portion are of course equal.

By having an end face 290 of the tubular mouthpiece portion 225 which is angled, the patient will be encouraged to tilt the actuator 200 backwards away from the face, with the spray being directed upwards with respect to their oral cavity. The end face 290 provides both a visual and tactile orientation clue for the correct positioning and alignment of an MDI device having an actuator housing as shown in FIG. 6.

FIG. 6 forms the basis for the embodiments described below with reference to FIGS. 7 to 14 in that each of these embodiments has a mouthpiece end face or mouthpiece face plane which is angled with respect to the axis of the tubular housing portion and with respect to a plane perpendicular to the axis of the tubular mouthpiece portion. It should be understood, however, that these additional features could also be incorporated in the other embodiments (shown in FIGS. 3A to 5A) described above. Additional features are provided in or on the mouthpiece portion to provide indications for a patient when using an MDI device incorporating an actuator housing according to each of these embodiments.

The actuator housings of FIGS. 7 to 13a and 13b have similarities to the conventional actuator 10 shown in FIGS. 1a and 1b and to the actuator in accordance with the embodiment shown in FIG. 6. Elements which have been previously described have the same reference numerals and will not be described again here.

FIGS. 7a and 7b illustrate an MDI actuator housing 300 having, in addition to the tubular mouthpiece portion 325 having an end face or mouthpiece face plane 390 which is angled with respect to the axis of the tubular housing portion and with respect to a plane perpendicular to the axis of the tubular mouthpiece portion, a set of annular rings 395 formed on an external surface of the mouthpiece portion 325 adjacent and spaced from the end face or mouthpiece face plane 390. Each annular ring is arranged to be substantially parallel with the end face or mouthpiece face plane 390.

The provision of the set of annular rings 395 provides an indication of the correct orientation of the actuator 300 for optimised usage, and the set of annular rings 395 prevents the patient from creating a seal with the mouthpiece portion 325 unless the actuator 300 is tilted at an angle.

In addition to encouraging the patient to use the MDI device using the actuator 300 at an angle, the set of annular rings 395 also provides a ledge over which the teeth of the patient can be located. Furthermore, because the annular rings provide an uncomfortable region onto which to seal the lips, the patient is intuitively encouraged to push the mouthpiece further into their oral cavity in order that their lips can be sealed against the smooth region of the mouthpiece tube beyond the rings. Because of the limited distance between the lips and the teeth, this helps to ensure that the patient must also place their teeth around the outside of the mouthpiece instead of positioning them across its open end where they can obstruct the emerging spray plume. This ensures that the mouth is fully open and the airway is clear.

Although the set of annular rings 395 is shown comprising three rings, it will be appreciated that any suitable number of rings may be provided.

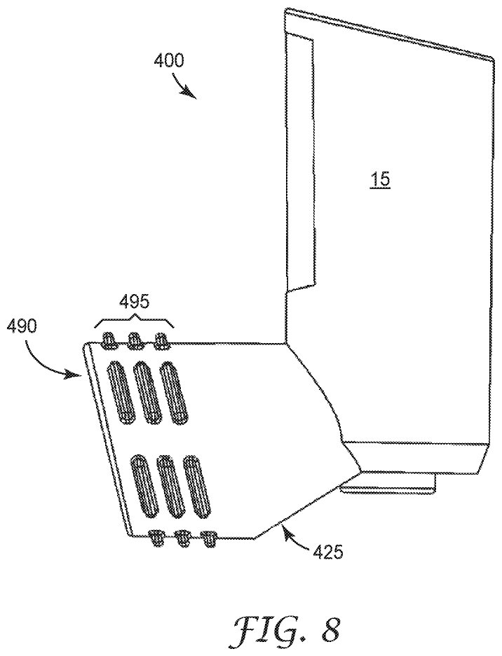

FIG. 8 is similar to FIG. 7b but illustrates an MDI actuator housing 400 in which a set of annular rings 495 are formed on an external surface of a tubular mouthpiece portion 425 adjacent and spaced from the end face or mouthpiece face plane 490 of the mouthpiece portion 425, and each annular ring of the set of annular rings 495 comprises a set of discontinuous elements which may further disincline the patient from placing their lips an insufficient distance along the mouthpiece. As described above, a seal will not be created unless all the elements are inside the mouth and the lips of the patient are wrapped around the tubular mouthpiece portion 425.

Although the discontinuous elements are shown as being regularly spaced on the tubular mouthpiece portion 425, it will readily be appreciated that these elements may also be irregularly spaced both around the tubular mouthpiece portion and extending away from the end face or mouthpiece face plane 490.

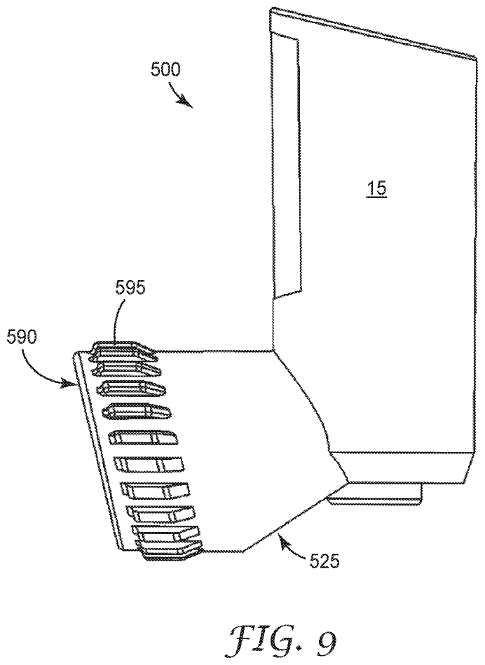

FIG. 9 illustrates an MDI actuator housing 500 having a tubular mouthpiece portion 525 having a set of rib elements 595 formed on an external surface thereof which are positioned adjacent and spaced from end face or mouthpiece face plane 590. Elements that have been described previously with reference to FIGS. 1a and 1b have the same reference numerals and are not described again here. The set of rib elements 595 is regularly spaced around the mouthpiece portion 525 and extends in a direction away from the end face or mouthpiece face plane 590. The set of ribbed elements 595 together with the end face or mouthpiece face plane 590 also align the actuator 500 in a correct orientation to ensure that a seal can be formed between lips of the patient and the tubular mouthpiece portion 525 when the actuator is inserted far enough into the mouth so that the lips can seal beyond the ends of the rib elements remote from the end face or mouthpiece face plane 590. The rib elements may be of any suitable shape and, in one embodiment, be shaped to provide a comfortable rest for the teeth of the patient so that the teeth are out of the airflow path between the exit orifice or actuator nozzle 45 of the nozzle block 30 located at the closed end 15b of the tubular housing portion 15 (FIG. 1a) and the end face or mouthpiece face plane 590.

Although, in the illustrated embodiment, the rib elements are regularly spaced around the tubular mouthpiece portion 525, it will readily be appreciated that the rib elements may be spaced in some other way (i.e., either regular or irregular spacing). For example, more rib elements may be provided on an upper portion of the external surface of the tubular mouthpiece portion for providing a location for the upper teeth with less rib elements being provided on a lower portion of the external surface of the tubular mouthpiece portion, and/or, the spacing between the rib elements may be variable around the tubular mouthpiece portion with the spaces between the rib elements on the upper surface being smaller than the spaces between the rib elements on the lower surface. Alternatively, the spacing between the rib elements may be variable around the tubular mouthpiece portion with the spaces between the rib elements on the upper surface being greater than the spaces between the rib elements on the lower surface.

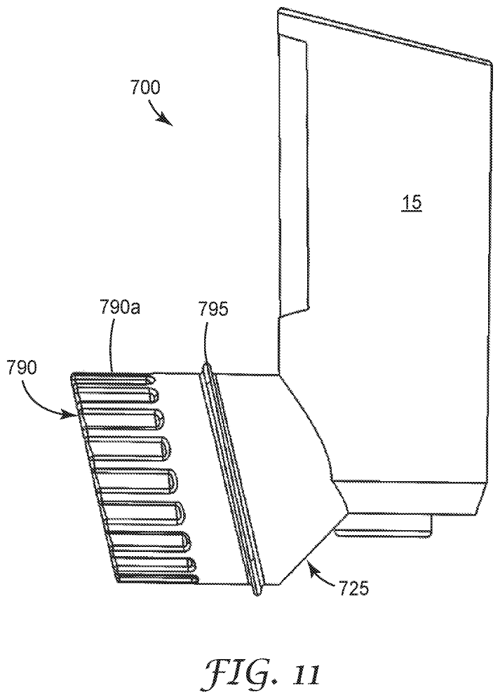

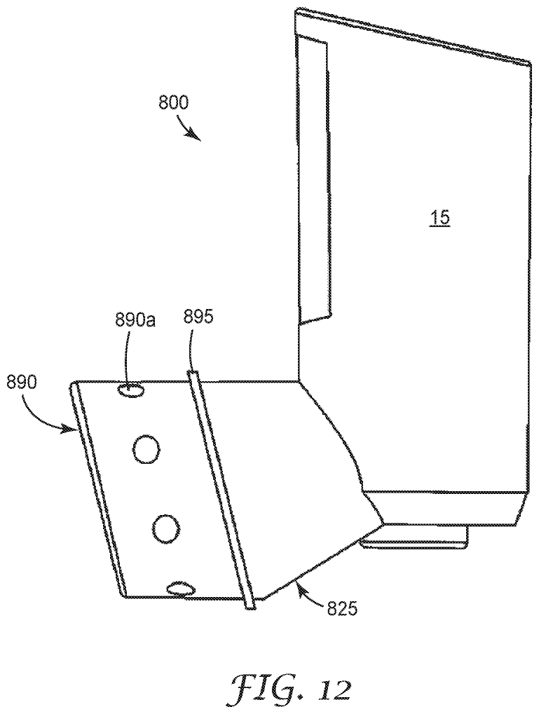

In addition, an annular projection may be provided (not shown) which limits the amount that the mouthpiece is inserted into the mouth as will be described in more detail below with reference to FIGS. 11 and 12.

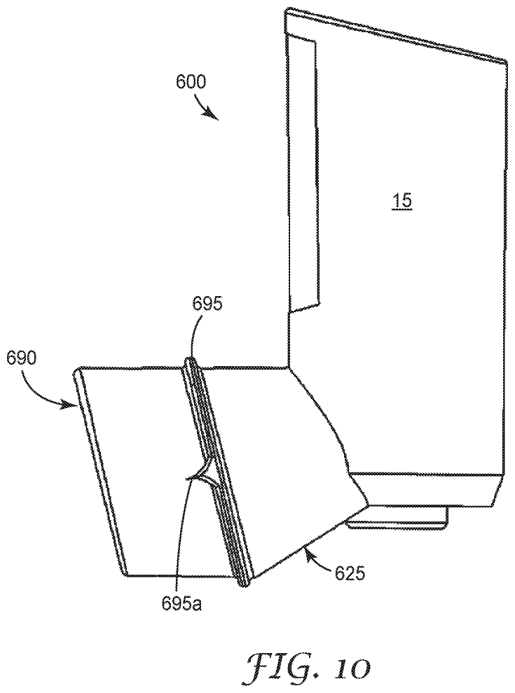

FIG. 10 illustrates an MDI actuator housing 600 having a tubular mouthpiece portion 625 having an end face or mouthpiece face plane 690. Elements that have been described previously with reference to FIGS. 1a and 1b have the same reference numerals and are not described again here. An annular ring 695 is provided on an external surface of the mouthpiece portion 625 and which is spaced from the end face or mouthpiece face plane 690, the annular ring 695 being shaped with pinched features 695a on each side to approximate the shape of the lips of the patient so that the patient has a guide against which to butt their lips. The butting of the lips with the annular ring 695 ensures that the mouthpiece portion 625 of the actuator housing 600 is inserted far enough into the mouth of the patient so that the teeth straddle the tubular mouthpiece portion 625 and are clear of the airflow path.

FIG. 11 illustrates an MDI actuator housing 700 which combines two features to prevent the patient from creating a seal unless the lips and mouth are far enough along tubular mouthpiece portion 725, and, to prevent the patient from inserting the mouthpiece portion of the actuator housing too far into the mouth. In this embodiment, the tubular mouthpiece portion 725 has an end face or mouthpiece face plane 790 in a similar way to the embodiments described with reference to FIGS. 6 to 10.

A fluted end 790a is provided in an external surface of the mouthpiece portion 725 adjacent the end face or mouthpiece face plane 790 together with an annular ring 795 spaced therefrom. The annular ring 795 is similar to one of the set of annular rings 395 of the actuator 300 (FIG. 7b) or to the annular ring 695 of the actuator 600 (FIG. 10) but not shaped to form a butt for the lips of a patient. However, it will be appreciated that the annular ring 795 may be similar to annular ring 695, 695a, that is, shaped to form a butt for the lips of the patient. In this embodiment, the annular ring 795 prevents the patient from inserting the actuator 700 too far into the mouth.

It will readily be appreciated that the end of the mouthpiece need not be fluted and that a series of projections, depressions or holes alternatively may be provided to prevent a seal being formed between the external surface of the mouthpiece portion and the lips of the patient.

In this way, the combination of a feature that ensures that the mouthpiece portion of the actuator housing is inserted far enough to be able to form a seal around the tubular mouthpiece portion, of a feature that encourages the actuator housing to be angled away from the face, and of a feature that prevents the mouthpiece portion of the actuator housing being inserted too far into the mouth provides a useful guide for a patient regarding the positioning and use of an MDI device incorporating such an actuator housing.

FIG. 12 illustrates an MDI actuator housing 800 which combines two features to prevent the patient from creating a seal unless the lips and mouth are far enough along tubular mouthpiece portion 825, and, to discourage the patient from inserting the mouthpiece portion of the actuator housing too far into the mouth. In this embodiment, the tubular mouthpiece portion 825 has an angled end face or mouthpiece face plane 890 in a similar way to the embodiments described with reference to FIGS. 6 to 11.

Adjacent the end face or mouthpiece face plane 890 is provided a ring of holes 890a together with an annular ring 895 spaced therefrom. The annular ring 895 prevents the patient from inserting the mouthpiece portion 825 of the actuator housing 800 too far into the mouth.

Although, in the illustrated embodiment, the holes are regularly spaced around the tubular mouthpiece portion 825, it will readily be appreciated that the holes may be spaced in some other way (i.e., either regular or irregular spacing). For example, more holes may be provided on an upper portion of the external surface of the tubular mouthpiece portion with less holes being provided on a lower portion of the external surface of the tubular mouthpiece portion, and/or, the spacing between the holes may be variable around the tubular mouthpiece portion

It will readily be appreciated that more than one ring of holes may be provided in the mouthpiece portion. The rings of holes may be aligned with one another such that each hole forms a line which extends parallel to the axis of the mouthpiece portion. The holes of one ring may also be arranged to interleave with holes of another ring to form a honeycomb-type arrangement in the tubular mouthpiece portion.

In a similar way to the embodiment of FIG. 11, the combination of a feature that ensures that the mouthpiece of the actuator housing is inserted far enough into the mouth to be able to form a seal around the tubular mouthpiece portion, of a feature that encourages that the housing portion of the actuator housing to be angled away from the face, and of a feature that prevents the mouthpiece of the actuator housing being inserted too far into the mouth provides a useful guide for a patient regarding the positioning and use of an MDI device incorporating such an actuator housing.

As an alternative to a ring of holes 890a, a ring of depressions may be provided adjacent to the end face or mouthpiece face plane (not shown) which ensures that the mouthpiece of the actuator housing is inserted far enough into the mouth to be able to form a seal around the tubular mouthpiece portion. The ring of depressions may define at least one ring with depressions of one ring being aligned with corresponding depressions of another ring in a direction which extends parallel to the axis of the mouthpiece portion. The depressions may be spaced (i.e., regular or irregular spacing) in the same manner as described above for holes. The depressions of one ring may also be arranged to interleave with depressions of another ring to form a honeycomb-type arrangement in the tubular mouthpiece portion. An annular projection may be provided to limit the amount of the mouthpiece portion inserted into the mouth of the patient as described above.

Naturally, the ring of holes 890a may be replaced by a ring of projections provided adjacent to the end face or mouthpiece face plane (not shown) which ensures that the mouthpiece of the actuator housing is inserted far enough into the mouth to be able to form a seal around the tubular mouthpiece portion. The ring of projections may define at least one ring and be arranged as described above. An annular projection may be provided to limit the amount of the mouthpiece portion which needs to be inserted into the mouth of the patient as described above.

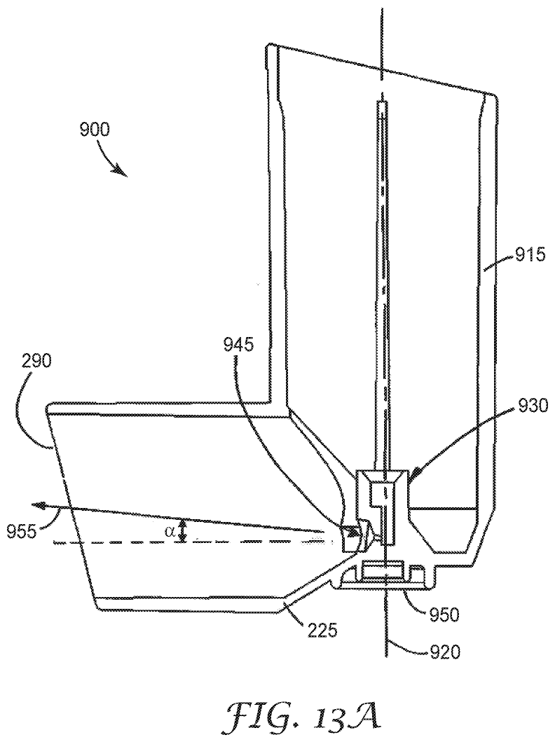

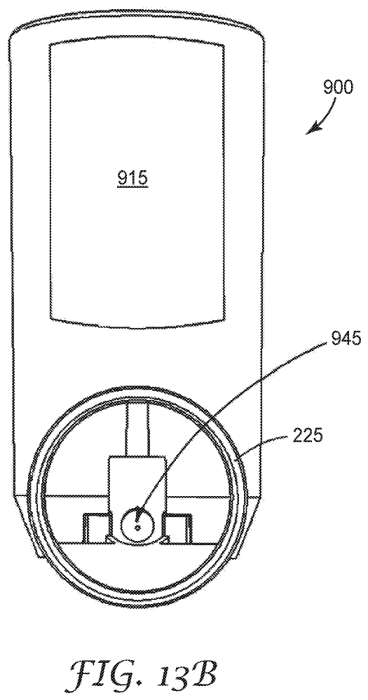

FIG. 13a illustrates a side sectioned view through another embodiment of an MDI actuator housing 900 in accordance with the present invention. The actuator housing 900 comprises a tubular housing portion 915 having an axis 920 extending therethrough and a tubular mouthpiece portion 225 which is the same as the tubular mouthpiece portion shown in FIG. 6. Elements that are the same as those described with reference to FIG. 6 are numbered exactly the same. FIG. 13b shows the actuator of FIG. 13a, viewed from the front.

Within the closed or proximal end of the tubular housing portion 915 sits a nozzle block 930 having an exit orifice or actuator nozzle 945, the nozzle block 930 being aligned with the axis 920. In this case, the nozzle block 930 is located nearer to the bottom or base portion of the tubular housing portion 915. The tubular mouthpiece portion 225 extends from the closed or proximal end of the tubular housing portion 915 in a direction which is substantially parallel with a plane in which the base portion and the thumb grip 950 lie. In the illustrated embodiment, this plane is a horizontal plane, but it will readily be appreciated that the plane may be orientated at any other angle in accordance with the orientation of the actuator housing 900.

However, in this embodiment, the exit orifice or actuator nozzle 945 is angled upwardly with respect to the base portion and thumb grip 950 so that a spray dispensed therefrom, as a plume centred along a spray axis (indicated by arrow 955) forms an angle α with respect to the horizontal plane defined by a plane in which the base portion and thumb grip lie. Angle α may be in the range of between 20 degrees and 50 degrees, more preferably between 20 degrees and 40 degrees, and most preferably between 25 degrees and 35 degrees. In particular, an upward angle of 30 degrees has been found to be most effective in reducing buccal deposition, and in particular, tongue deposition.

The embodiment of FIGS. 13a, 13b not only causes the patient to angle the tubular mouthpiece 225 in the mouth with the housing portion angled away from the face when using an MDI device including such an actuator housing, but the upward angling of the spray by angle α ensures that the spray passes over the tongue of the patient and into the airway.

The lowering of the nozzle block 930, and in particular, the exit orifice or actuator nozzle 945 reduces deposition of the medicament on the uppermost inner surface of the tubular mouthpiece portion 225. Ideally, the spray is aligned with a line between a centre of the exit orifice or actuator nozzle 945 and a centre of end face or mouthpiece face plane 290 of the tubular mouthpiece portion 225.

It will be appreciated that it is possible to combine any one of the features described with reference to the embodiments of FIGS. 7 to 12 with an upwardly angled exit orifice or actuator nozzle as described with reference to FIGS. 13a and 13b.

1. An actuator housing for a metered dose inhaler device, the actuator housing comprising:
a substantially hollow tubular first portion having a first proximal end and a first distal end;
a base portion formed at the first proximal end, the base portion having a base plane;
an actuator seat formed in the base portion;
an actuator nozzle formed in the actuator seat and operable for dispensing a spray of metered fluid; and
a substantially hollow second portion having a second proximal end and a second distal end and defining a mouthpiece, the second proximal end being located adjacent the first proximal end and the second distal end defining an end of the mouthpiece, the substantially hollow second portion defining a roof section and a floor section which extend from the second proximal end to the second distal end of the mouthpiece, the roof section having an outer profile angled upwardly relative to the base plane and the floor section being in juxtaposition with the base portion and having an outer profile substantially parallel with the base plane.

2. An actuator housing according to claim 1, wherein the outer profile of the roof section defines an upward angle between 10 degrees and 40 degrees relative to the base plane.

3. (canceled)

4. An actuator housing according to claim 1, wherein the actuator nozzle is upwardly angled with respect to the base plane to produce an upwardly angled spray with respect to the base plane.

5. An actuator housing according to claim 4, wherein the actuator seat is angled with respect to the base plane to produce the upwardly angled actuator nozzle.

6. (canceled)

7. An actuator housing according to claim 1, further comprising a cross-piece formed at a first end of the outer profile of the roof section which is adjacent the second proximal end of the mouthpiece.

8. An actuator housing for a metered dose inhaler device, the actuator housing comprising:
a substantially hollow tubular first portion having a first proximal end and a first distal end and a first portion axis extending from the first distal end to the first proximal end;
a base portion formed at the first proximal end;
an actuator seat formed in the base portion;
an actuator nozzle formed in the actuator seat and operable for dispensing a spray of metered fluid; and
a substantially hollow tubular second portion having a second proximal end and a second distal end defining a mouthpiece, the second proximal end being located adjacent the first proximal end and the second distal end defining an end face of the mouthpiece, the substantially hollow tubular second portion having a second portion axis extending from a centre of the actuator nozzle to a centre of the end face of the mouthpiece;
wherein the actuator nozzle defines a spray axis which is angled upwardly in the distal direction relative to a plane perpendicular to the first portion axis.

9. An actuator housing according to claim 8, wherein the second portion axis is angled upwardly relative to a plane perpendicular to the first portion axis.

10. An actuator housing according to claim 9, wherein the spray axis is aligned with the second portion axis.

11. An actuator housing according to claim 8, wherein the spray axis is upwardly angled in the distal direction relative to the second portion axis.

12. An actuator housing according to claim 8, wherein the spray axis is angled upwardly between 10 degrees and 50 degrees relative to the plane perpendicular to the first portion axis.

13. (canceled)

14. An actuator housing for a metered dose inhaler device, the actuator housing comprising:
a generally tubular substantially hollow first portion having a first distal end and a first proximal end and a first portion axis extending from the first distal end to the first proximal end;
a base portion formed at the first proximal end;
an actuator seat formed in the base portion;
an actuator nozzle formed in the actuator seat and operable for dispensing a spray of metered fluid; and
a generally tubular substantially hollow second portion having a second proximal end and a second distal end defining a mouthpiece, the second proximal end being located adjacent the first proximal end and the second distal end defining an end of the mouthpiece, the generally tubular substantially hollow second portion having a second portion axis extending from a centre of the actuator nozzle to a centre of the end of the mouthpiece, the end of the mouthpiece having an open face defining a mouthpiece face plane which is angled relative to a plane perpendicular to the second portion axis, the substantially hollow second portion defining a floor portion being in juxtaposition with the base portion and a roof portion and, wherein the mouthpiece face plane is angled such that the roof portion extends distally, in the second portion axial direction, further than the floor portion.

15. An actuator housing according to claim 14, wherein the mouthpiece face plane is at an angle between 10 degrees and 50 degrees relative to a plane perpendicular to the second portion axis.

16. (canceled)

17. An actuator housing according to claim 14, wherein the actuator nozzle defines a spray axis which is angled upwardly in the distal direction relative to the second portion axis.

18. An actuator housing according to claim 14, further comprising a plurality of flutes formed on an external surface of the mouthpiece and located adjacent to the mouthpiece face plane.

19-20. (canceled)

21. An actuator housing according to claim 14, further comprising a plurality of apertures formed in the mouthpiece adjacent the mouthpiece face plane.

22-23. (canceled)

24. An actuator housing according to claim 14, further comprising a plurality of depressions formed on the mouthpiece adjacent the mouthpiece face plane.

25-26. (canceled)

27. An actuator housing according to claim 14, further comprising a plurality of rib elements formed on an external surface of the mouthpiece adjacent the mouthpiece end plane.

28-30. (canceled)

31. An actuator housing according to claim 14, wherein the mouthpiece includes at least one annular projection formed on an external surface thereof, each annular projection being spaced from the mouthpiece face plane.

32-36. (canceled)

37. An actuator housing according to claim 14, wherein the actuator nozzle is upwardly angled with respect to the base portion to produce an upwardly angled spray with respect thereto.

38. A metered dose inhaler device comprising a canister having a metering valve, and an actuator housing according to claim 1, the metering valve being operable for engaging with the actuator seat formed in the base portion of the actuator housing.

39-44. (canceled)
